(19) United States
(12) Patent Application Publication
(10) Pub. No.: US 2025/0009044 A1
(43) Pub. Date: Jan. 9, 2025

(54) AEROSOL PROVISION SYSTEM

(71) Applicant: NICOVENTURES TRADING LIMITED, London (GB)

(72) Inventor: Jeremy CAMPBELL, London (GB)

(21) Appl. No.: 18/708,855

(22) PCT Filed: Oct. 24, 2022
(86) PCT No.: PCT/GB2022/052706

§ 371 Date: May 9, 2024

(30) Foreign Application Priority Data
Nov. 10, 2021 (GB) 2116145.0

(51) Int. Cl. A24F 40/65; A24F 40/50

(52) U.S. Cl. CPC A24F 40/65; A24F 40/50

(57) ABSTRACT

A method for an aerosol provision system 1, wherein the method comprises: generating data, such as usage data, from the aerosol provision system 1. The data 290 is then wirelessly transmitted by the aerosol provision system 1, as part of a wireless signal, and is received at a third party location 200. Such a third party location could be an electrical or portable device 200A belonging to a relative of the user of the aerosol provision system 1. In this way, the relative, or other third party, can potentially send back a response signal to the aerosol provision system 1 for controlling an operation of the aerosol provision system 1.

### FIELD

The present disclosure relates to aerosol provision systems such as, but not limited to, nicotine delivery systems (e.g. electronic cigarettes and the like).

### BACKGROUND

Electronic aerosol provision systems often employ an electronic cigarette (e-cigarette) or more generally an aerosol provision device. Such an aerosol provision system typically contains aerosolisable material (also called aerosol-generating material), such as a reservoir of fluid or liquid containing a formulation, typically but not necessarily including nicotine, or a solid material such as a tobacco-based product, from which a vapour/aerosol is generated for inhalation by a user, for example through heat vaporisation. Thus, an aerosol provision system will typically comprise a vaporiser (also called an aerosol generator), e.g. a heating element, arranged to aerosolise a portion of aerosolisable material to generate a vapour.

Once a vapour has been generated, the vapour may be passed through flavouring material to add flavour to the vapour (if the aerosolisable material was not itself flavoured), after which the (flavoured) vapour may be then delivered to a user via a mouthpiece from the aerosol provision system.

A potential drawback of a number of existing aerosol provision systems and associated aerosol provision devices is in respect of their inability to be appropriately monitored by entities or relatives that are not the user of the aerosol provision system, who may otherwise need access or visibility of how the aerosol provision system is being employed or operated. Various approaches are therefore described herein which seek to help address or mitigate some of these issues, through the provision of data, such as usage data, which is configured to be transmitted from the aerosol provision system, and received at a third party location. Also provided herein are mechanisms for implementing, and selectively controlling, how and when this data is transmitted by the aerosol provision system.

### SUMMARY

According to a first aspect of certain embodiments there is provided a method for an aerosol provision system, wherein the method comprises:
generating data from the aerosol provision system;
wirelessly transmitting the data to a third party location; and
receiving the data at the third party location.

According to a second aspect of certain embodiments there is provided a communication system comprising:
an aerosol provision system, for generating an aerosol, and configured to wirelessly transmit data in a first mode of operation; and
a device, remotely located from the aerosol provision system, wherein the device is configured to transmit a control signal to the aerosol provision system for instructing the aerosol provision system to change between:
   the first mode of operation in which the aerosol provision system is configured to wirelessly transmit data; and
   a second mode of operation in which the aerosol provision system is configured to not wirelessly transmit the data.

According to a third aspect of certain embodiments there is provided a communication system comprising:
an aerosol provision system, for generating an aerosol, and configured to generate, and wireless transmit, data; and
a third party location, wherein the third party location is configured to receive the data from the aerosol provision system.

According to a fourth aspect of certain embodiments there is provided an aerosol provision system for generating an aerosol, wherein the aerosol provision system is configured to:
operate in a first mode of operation in which the aerosol provision system is configured to wirelessly transmit data to a predetermined third party location; and
operate in a second mode of operation in which the aerosol provision system is configured to not wirelessly transmit data to the predetermined third party location.

It will be appreciated that features and aspects of the invention described above in relation to the various aspects of the invention are equally applicable to, and may be combined with, embodiments of the invention according to other aspects of the invention as appropriate, and not just in the specific combinations described herein.

### BRIEF DESCRIPTION OF THE DRAWINGS

Embodiments of the invention will now be described, by way of example only, with reference to the accompanying drawings, in which:

FIG. 1 schematically represents in perspective view an aerosol provision system comprising a cartridge and aerosol provision device (shown separated) in accordance with certain embodiments of the disclosure;

FIG. 2 schematically represents in exploded perspective view of components of the cartridge of the aerosol provision system of FIG. 1;

FIGS. 3A to 3C schematically represent various cross-section views of a housing part of the cartridge of the aerosol provision system of FIG. 1;

FIGS. 4A and 4B schematically represent a perspective view and a plan view of a dividing wall element of the cartridge of the aerosol provision system of FIG. 1;

FIGS. 5A to 5C schematically represent two perspective views and a plan view of a resilient plug of the cartridge of the aerosol provision system of FIG. 1;

FIGS. 6A and 6B schematically represent a perspective view and a plan view of a bottom cap of the cartridge of the aerosol provision system of FIG. 1;

FIG. 7 schematically represents embodiments of communication system, useable with an aerosol provision system such as that shown in FIGS. 1-6B, and comprising a third party location (or third party device) which is configured to receive data, such as by a wireless signal, which is transmitted from the aerosol provision system;

FIG. 8 schematically represents an embodiment of the communication system from FIG. 7, whereby the third party location comprises an electrical device which does not belong to the user of the aerosol provision system, such as instead an electrical device belonging to a friend, or family member of the user of the aerosol provision system, in accordance with certain embodiments of the disclosure;

FIG. 9 schematically represents an embodiment of the communication system from FIG. 7, whereby the third party location comprises a different form of third party location, such as a health provider, or a manufacturer of the aerosol provision system, in accordance with certain embodiments of the disclosure; and

FIG. 10 schematically represents a method of selectively controlling the aerosol provision system to operate in a first mode of operation in which the aerosol provision system is configured to wirelessly transmit data to the third party location, and/or operate in a second mode of operation in which the aerosol provision system is configured to not wirelessly transmit data to the third party location, in accordance with certain embodiments of the disclosure.

### DETAILED DESCRIPTION

Aspects and features of certain examples and embodiments are discussed/described herein. Some aspects and features of certain examples and embodiments may be implemented conventionally and these are not discussed/described in detail in the interests of brevity. It will thus be appreciated that aspects and features of apparatus and methods discussed herein which are not described in detail may be implemented in accordance with any conventional techniques for implementing such aspects and features.

The present disclosure relates to non-combustible aerosol provision systems (such as an e-cigarette). According to the present disclosure, a “non-combustible” aerosol provision system is one where a constituent aerosolisable material of the aerosol provision system (or component thereof) is not combusted or burned in order to facilitate delivery to a user. Aerosolisable material, which also may be referred to herein as aerosol generating material or aerosol precursor material, is material that is capable of generating aerosol, for example when heated, irradiated or energized in any other way. The aerosolisable material may also be flavoured, in some embodiments.

Throughout the following description the term “e-cigarette” or “electronic cigarette” may sometimes be used, but it will be appreciated this term may be used interchangeably with an aerosol provision system. An electronic cigarette may also known as a vaping device or electronic nicotine delivery system (END), although it is noted that the presence of nicotine in the aerosolisable material is not a requirement.

In some embodiments, the aerosol provision system is a hybrid device configured to generate aerosol using a combination of aerosolisable materials, one or a plurality of which may be heated. In some embodiments, the hybrid device comprises a liquid or gel aerosolisable material and a solid aerosolisable material. The solid aerosolisable material may comprise, for example, tobacco or a non-tobacco product.

Typically, the (non-combustible) aerosol provision system may comprise a cartridge/consumable part and a body/reusable/aerosol provision device part, which is configured to releasably engage with the cartridge/consumable part.

The aerosol provision system may be provided with a means for powering a vaporiser therein, and there may be provided an aerosolisable material transport element for receiving the aerosolisable material that is to be vaporised. The aerosol provision system may also be provided with a reservoir for containing aerosolisable material, and in some embodiments a further reservoir for containing flavouring material for flavouring a generated vapour from the aerosol provision system.

In some embodiments, the vaporiser may be a heater/heating element capable of interacting with the aerosolisable material so as to release one or more volatiles from the aerosolisable material to form a vapour/aerosol. In some embodiments, the vaporiser is capable of generating an aerosol from the aerosolisable material without heating. For example, the vaporiser may be capable of generating a vapour/aerosol from the aerosolisable material without applying heat thereto, for example via one or more of vibrational, mechanical, pressurisation or electrostatic means.

In some embodiments, the substance to be delivered may be an aerosolisable material which may comprise an active constituent, a carrier constituent and optionally one or more other functional constituents.

The active constituent may comprise one or more physiologically and/or olfactory active constituents which are included in the aerosolisable material in order to achieve a physiological and/or olfactory response in the user. The active constituent may for example be selected from nutraceuticals, nootropics, and psychoactives. The active constituent may be naturally occurring or synthetically obtained. The active constituent may comprise for example nicotine, caffeine, taurine, theine, a vitamin such as B6 or B12 or C, melatonin, a cannabinoid, or a constituent, derivative, or combinations thereof. The active constituent may comprise a constituent, derivative or extract of tobacco or of another botanical. In some embodiments, the active constituent is a physiologically active constituent and may be selected from nicotine, nicotine salts (e.g. nicotine ditartrate/nicotine bitartrate), nicotine-free tobacco substitutes, other alkaloids such as caffeine, or mixtures thereof.

In some embodiments, the active constituent is an olfactory active constituent and may be selected from a “flavour” and/or “flavourant” which, where local regulations permit, may be used to create a desired taste, aroma or other somatosensorial sensation in a product for adult consumers. In some instances such constituents may be referred to as flavours, flavourants, flavouring material, cooling agents, heating agents, and/or sweetening agents. They may include naturally occurring flavour materials, botanicals, extracts of botanicals, synthetically obtained materials, or combinations thereof (e.g., tobacco, cannabis, licorice (liquorice), hydrangea, eugenol, Japanese white bark magnolia leaf, chamomile, fenugreek, clove, maple, matcha, menthol, Japanese mint, aniseed (anise), cinnamon, turmeric, Indian spices, Asian spices, herb, wintergreen, cherry, berry, red berry, cranberry, peach, apple, orange, mango, clementine, lemon, lime, tropical fruit, papaya, rhubarb, grape, durian, dragon fruit, cucumber, blueberry, mulberry, citrus fruits, Drambuie, bourbon, scotch, whiskey, gin, tequila, rum, spearmint, peppermint, lavender, aloe vera, cardamom, celery, cascarilla, nutmeg, sandalwood, bergamot, geranium, khat, naswar, betel, shisha, pine, honey essence, rose oil, vanilla, lemon oil, orange oil, orange blossom, cherry blossom, cassia, caraway, cognac, jasmine, ylang-ylang, sage, fennel, wasabi, piment, ginger, coriander, coffee, hemp, a mint oil from any species of the genus Mentha, eucalyptus, star anise, cocoa, lemongrass, rooibos, flax, Ginkgo biloba, hazel, hibiscus, laurel, mate, orange skin, rose, tea such as green tea or black tea, thyme, juniper, elderflower, basil, bay leaves, cumin, oregano, paprika, rosemary, saffron, lemon peel, mint, beefsteak plant, curcuma, cilantro, myrtle, cassis, valerian, pimento, mace, damien, marjoram, olive, lemon balm, lemon basil, chive, carvi, verbena, tarragon, limonene, thymol, camphene), flavour enhancers, bitterness receptor site blockers, sensorial receptor site activators or stimulators, sugars and/or sugar substitutes (e.g., sucralose, acesulfame potassium, aspartame, saccharine, cyclamates, lactose, sucrose, glucose, fructose, sorbitol, or mannitol), and other additives such as charcoal, chlorophyll, minerals, botanicals, or breath freshening agents. They may be imitation, synthetic or natural ingredients or blends thereof. They may be in any suitable form, for example, liquid such as an oil, solid such as a powder, or gasone or more of extracts (e.g., licorice, hydrangea, Japanese white bark magnolia leaf, chamomile, fenugreek, clove, menthol, Japanese mint, aniseed, cinnamon, herb, wintergreen, cherry, berry, peach, apple, Drambuie, bourbon, scotch, whiskey, spearmint, peppermint, lavender, cardamom, celery, cascarilla, nutmeg, sandalwood, bergamot, geranium, honey essence, rose oil, vanilla, lemon oil, orange oil, cassia, caraway, cognac, jasmine, ylang-ylang, sage, fennel, piment, ginger, anise, coriander, coffee, or a mint oil from any species of the genus Mentha), flavour enhancers, bitterness receptor site blockers, sensorial receptor site activators or stimulators, sugars and/or sugar substitutes (e.g., sucralose, acesulfame potassium, aspartame, saccharine, cyclamates, lactose, sucrose, glucose, fructose, sorbitol, or mannitol), and other additives such as charcoal, chlorophyll, minerals, botanicals, or breath freshening agents. They may be imitation, synthetic or natural ingredients or blends thereof. They may be in any suitable form, for example, oil, liquid, or powder.

In some embodiments, the flavouring material (flavour) may comprise menthol, spearmint and/or peppermint. In some embodiments, the flavour comprises flavour components of cucumber, blueberry, citrus fruits and/or redberry. In some embodiments, the flavour comprises eugenol. In some embodiments, the flavour comprises flavour components extracted from tobacco. In some embodiments, the flavour may comprise a sensate, which is intended to achieve a somatosensorial sensation which are usually chemically induced and perceived by the stimulation of the fifth cranial nerve (trigeminal nerve), in addition to or in place of aroma or taste nerves, and these may include agents providing heating, cooling, tingling, numbing effect. A suitable heat effect agent may be, but is not limited to, vanillyl ethyl ether and a suitable cooling agent may be, but not limited to eucalyptol, WS-3.

The carrier constituent may comprise one or more constituents capable of forming an aerosol. In some embodiments, the carrier constituent may comprise one or more of glycerine, glycerol, propylene glycol, diethylene glycol, triethylene glycol, tetraethylene glycol, 1,3-butylene glycol, erythritol, meso-Erythritol, ethyl vanillate, ethyl laurate, a diethyl suberate, triethyl citrate, triacetin, a diacetin mixture, benzyl benzoate, benzyl phenyl acetate, tributyrin, lauryl acetate, lauric acid, myristic acid, and propylene carbonate.

The one or more other functional constituents may comprise one or more of pH regulators, colouring agents, preservatives, binders, fillers, stabilizers, and/or antioxidants.

As noted above, aerosol provision systems (e-cigarettes) may often comprise a modular assembly including both a reusable part (body—or aerosol provision device) and a replaceable consumable (cartridge) part. Devices conforming to this type of two-part modular configuration may generally be referred to as two-part devices. It is also common for electronic cigarettes to have a generally elongate shape. For the sake of providing a concrete example, certain embodiments of the disclosure described herein may comprise this kind of generally elongate two-part device employing consumable parts. However, it will be appreciated the underlying principles described herein may equally be adopted for other electronic cigarette configurations, for example modular devices comprising more than two parts, as devices conforming to other overall shapes, for example based on so-called box-mod high performance devices that typically have a more boxy shape.

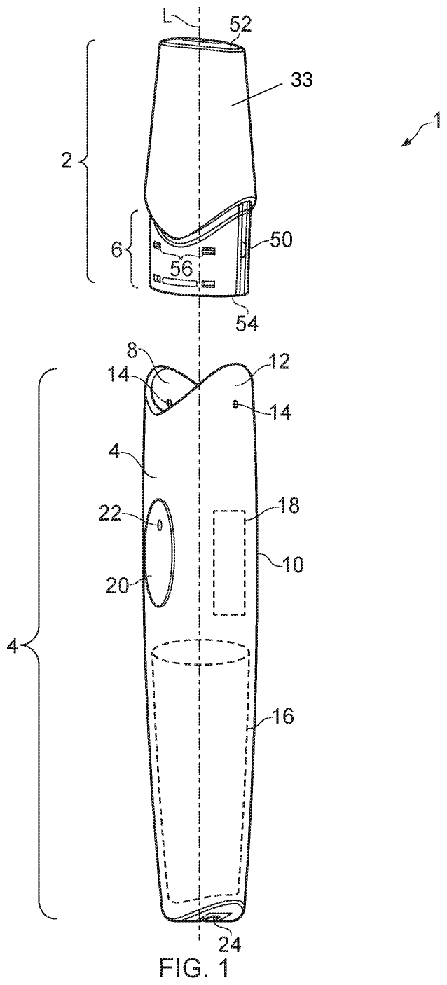

From the forgoing therefore, and with reference to FIG. 1 is a schematic perspective view of an example aerosol provision system (e-cigarette) 1 in accordance with certain embodiments of the disclosure. Terms concerning the relative location of various aspects of the electronic cigarette (e.g. terms such as upper, lower, above, below, top, bottom etc.) are used herein with reference to the orientation of the electronic cigarette as shown in FIG. 1 (unless the context indicates otherwise). However, it will be appreciated this is purely for ease of explanation and is not intended to indicate there is any required orientation for the electronic cigarette in use.

The e-cigarette 1 (aerosol provision system 1) comprises two main components, namely a cartridge 2 and an aerosol provision device 4. The aerosol provision device 4 and the cartridge 2 are shown separated in FIG. 1, but are coupled together when in use.

The cartridge 2 and aerosol provision device 4 are coupled by establishing a mechanical and electrical connection between them. The specific manner in which the mechanical and electrical connection is established is not of primary significance to the principles described herein and may be established in accordance with conventional techniques, for example based around a screw thread, bayonet, latched or friction-fit mechanical fixing with appropriately arranged electrical contacts/electrodes for establishing the electrical connection between the two parts as appropriate. For example electronic cigarette 1 represented in FIG. 1, the cartridge comprises a mouthpiece 33, a mouthpiece end 52 and an interface end 54 and is coupled to the aerosol provision device by inserting an interface end portion 6 at the interface end of the cartridge into a corresponding receptacle 8/receiving section of the aerosol provision device. The interface end portion 6 of the cartridge is a close fit to be receptacle 8 and includes protrusions 56 which engage with corresponding detents in the interior surface of a receptacle wall 12 defining the receptacle 8 to provide a releasable mechanical engagement between the cartridge and the aerosol provision device. An electrical connection is established between the aerosol provision device and the cartridge via a pair of electrical contacts on the bottom of the cartridge (not shown in FIG. 1) and corresponding sprung contact pins in the base of the receptacle 8 (not shown in FIG. 1). As noted above, the specific manner in which the electrical connection is established is not significant to the principles described herein, and indeed some implementations might not have an electrical connection between the cartridge and a aerosol provision device at all, for example because the transfer of electrical power from the reusable part to the cartridge may be wireless (e.g. based on electromagnetic induction techniques).

The electronic cigarette 1 (aerosol provision system) has a generally elongate shape extending along a longitudinal axis L. When the cartridge is coupled to the aerosol provision device, the overall length of the electronic cigarette in this example (along the longitudinal axis) is around 12.5 cm. The overall length of the aerosol provision device is around 9 cm and the overall length of the cartridge is around 5 cm (i.e. there is around 1.5 cm of overlap between the interface end portion 6 of the cartridge and the receptacle 8 of the aerosol provision device when they are coupled together). The electronic cigarette has a cross-section which is generally oval and which is largest around the middle of the electronic cigarette and tapers in a curved manner towards the ends. The cross-section around the middle of the electronic cigarette has a width of around 2.5 cm and a thickness of around 1.7 cm. The end of the cartridge has a width of around 2 cm and a thickness of around 0.6 mm, whereas the other end of the electronic cigarette has a width of around 2 cm and a thickness of around 1.2 cm. The outer housing of the electronic cigarette is in this example is formed from plastic. It will be appreciated the specific size and shape of the electronic cigarette and the material from which it is made is not of primary significance to the principles described herein and may be different in different implementations. That is to say, the principles described herein may equally be adopted for electronic cigarettes having different sizes, shapes and/or materials.

The aerosol provision device 4 may in accordance with certain embodiments of the disclosure be broadly conventional in terms of its functionality and general construction techniques. In the example of FIG. 1, the aerosol provision device 4 comprises a plastic outer housing 10 including the receptacle wall 12 that defines the receptacle 8 for receiving the end of the cartridge as noted above. The outer housing 10 of the aerosol provision device 4 in this example has a generally oval cross section conforming to the shape and size of the cartridge 2 at their interface to provide a smooth transition between the two parts. The receptacle 8 and the end portion 6 of the cartridge 2 are symmetric when rotated through 180° so the cartridge can be inserted into the aerosol provision device in two different orientations. The receptacle wall 12 includes two aerosol provision device air inlet openings 14 (i.e. holes in the wall). These openings 14 are positioned to align with an air inlet 50 for the cartridge when the cartridge is coupled to the aerosol provision device. A different one of the openings 14 aligns with the air inlet 50 of the cartridge in the different orientations. It will be appreciated some implementations may not have any degree of rotational symmetry such that the cartridge is couplable to the aerosol provision device in only one orientation while other implementations may have a higher degree of rotational symmetry such that the cartridge is couplable to the aerosol provision device in more orientations.

The aerosol provision device further comprises a battery 16 for providing operating power for the electronic cigarette, control circuitry 18 for controlling and monitoring the operation of the electronic cigarette, a user input button 20, an indicator light 22, and a charging port 24.

The battery 16 in this example is rechargeable and may be of a conventional type, for example of the kind normally used in electronic cigarettes and other applications requiring provision of relatively high currents over relatively short periods. The battery 16 may be recharged through the charging port 24, which may, for example, comprise a USB connector.

The input button 20 in this example is a conventional mechanical button, for example comprising a sprung mounted component which may be pressed by a user to establish an electrical contact in underlying circuitry. In this regard, the input button may be considered an input device for detecting user input, e.g. to trigger aerosol generation, and the specific manner in which the button is implemented is not significant. For example, other forms of mechanical button or touch-sensitive button (e.g. based on capacitive or optical sensing techniques) may be used in other implementations, or there may be no button and the device may rely on a puff detector for triggering aerosol generation.

The indicator light 22 is provided to give a user with a visual indication of various characteristics associated with the electronic cigarette, for example, an indication of an operating state (e.g. on/off/standby), and other characteristics, such as battery life or fault conditions. Different characteristics may, for example, be indicated through different colours and/or different flash sequences in accordance with generally conventional techniques.

The control circuitry 18 is suitably configured/programmed to control the operation of the electronic cigarette to provide conventional operating functions in line with the established techniques for controlling electronic cigarettes. The control circuitry (processor circuitry) 18 may be considered to logically comprise various sub-units/circuitry elements associated with different aspects of the electronic cigarette's operation. For example, depending on the functionality provided in different implementations, the control circuitry 18 may comprises power supply control circuitry for controlling the supply of power from the battery/power supply to the cartridge in response to user input, user programming circuitry for establishing configuration settings (e.g. user-defined power settings) in response to user input, as well as other functional units/circuitry associated functionality in accordance with the principles described herein and conventional operating aspects of electronic cigarettes, such as indicator light display driving circuitry and user input detection circuitry. It will be appreciated the functionality of the control circuitry 18 can be provided in various different ways, for example using one or more suitably programmed programmable computer(s) and/or one or more suitably configured application-specific integrated circuit(s)/circuitry/chip(s)/chipset(s) configured to provide the desired functionality.

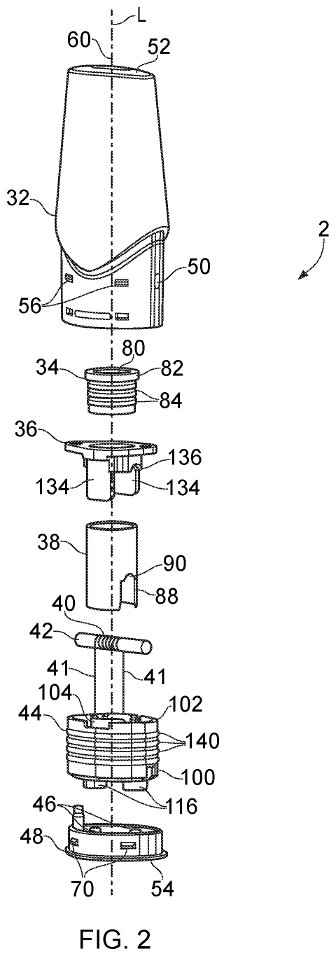

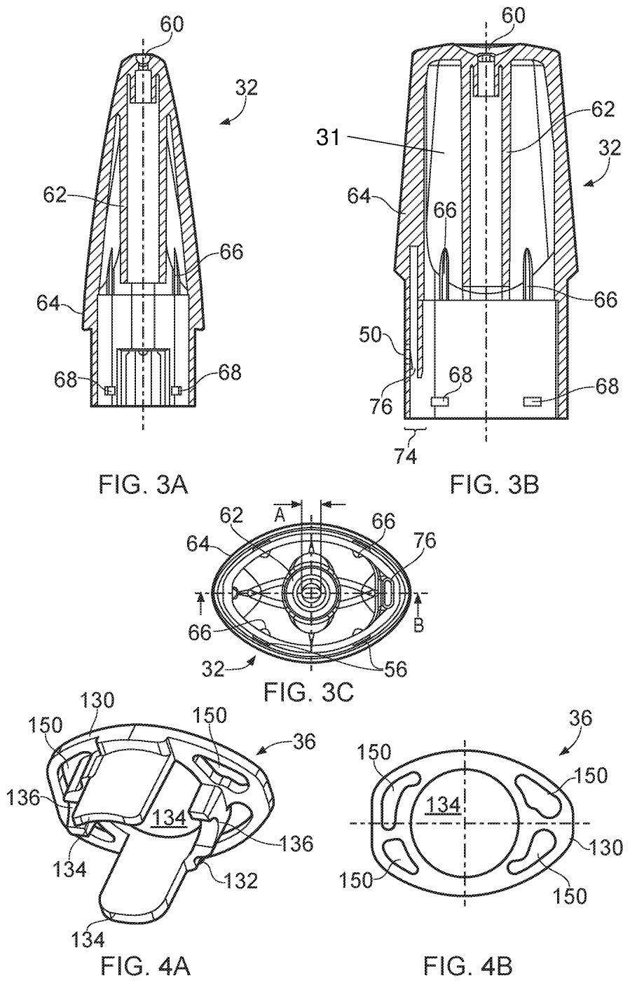

FIG. 2 is an exploded schematic perspective view of the cartridge 2 (exploded along the longitudinal axis L). The cartridge 2 comprises a housing part 32, an air channel seal 34, a dividing wall element 36, an outlet tube 38, a vaporiser/heating element 40, an aerosolisable material transport element 42, a plug 44, and an end cap 48 with contact electrodes 46. FIGS. 3 to 6 schematically represents some of these components in more detail.

FIG. 3A is a schematic cut-away view of the housing part 32 through the longitudinal axis L where the housing part 32 is thinnest. FIG. 3B is a schematic cut-away view of the housing part 32 through the longitudinal axis L where the housing part 32 is widest. FIG. 3C is a schematic view of the housing part along the longitudinal axis L from the interface end 54 (i.e. viewed from below in the orientation of FIGS. 3A and 3B).

FIG. 4A is a schematic perspective view of the dividing wall element 36 as seen from below. FIG. 4B is a schematic cross-section through an upper part of the dividing wall element 36 as viewed from below.

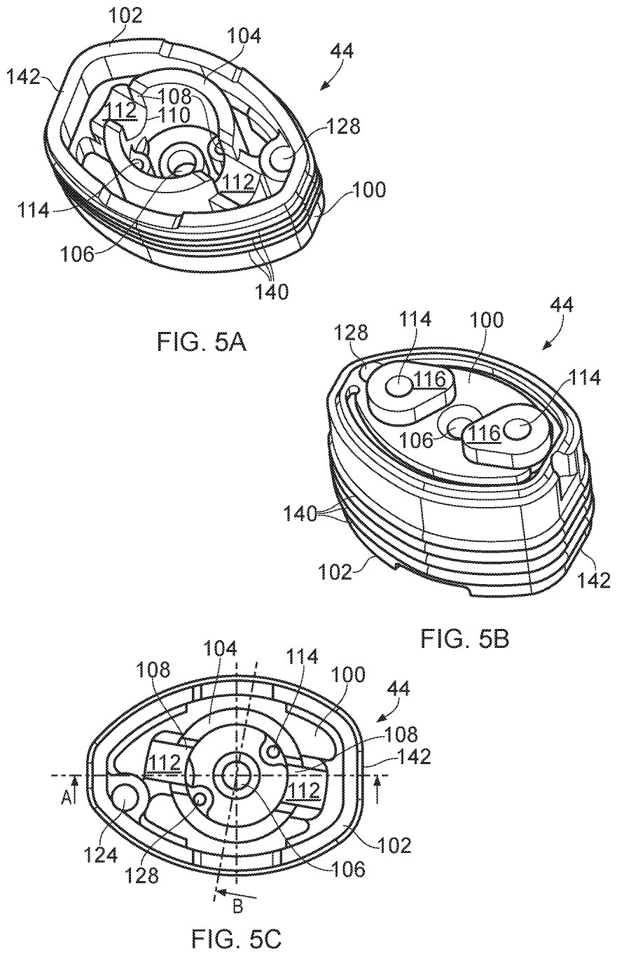

FIG. 5A is a schematic perspective view of the plug 44 from above and FIG. 5B is a schematic perspective view of the plug 44 from below. FIG. 5C is a schematic view of the plug 44 along the longitudinal axis L seen from the mouthpiece end 52 of the cartridge (i.e. viewed from above for the orientation in FIGS. 1 and 2).

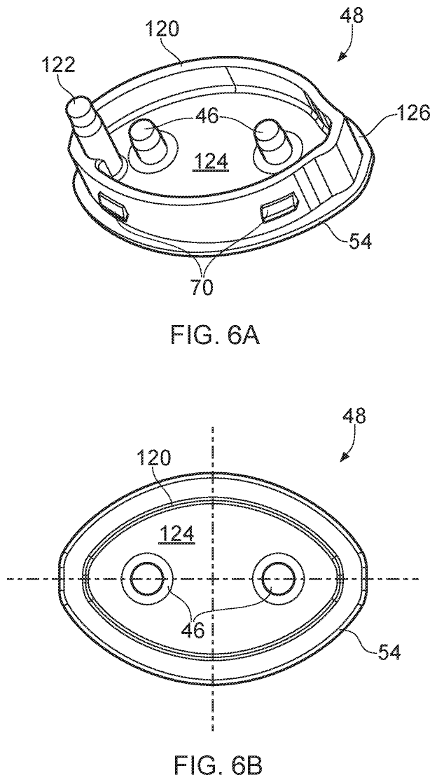

FIG. 6A is a schematic perspective view of the end cap 48 from above. FIG. 6B is a schematic view of the end cap 48 along the longitudinal axis L seen from the mouthpiece end 52 of the cartridge (i.e. from above).

The housing part 32 in this example comprises a housing outer wall 64 and a housing inner tube 62 which in this example are formed from a single moulding of polypropylene. The housing outer wall 64 defines the external appearance of the cartridge 2 and the housing inner tube 62 defines a part the air channel through the cartridge. The housing part is open at the interface end 54 of the cartridge and closed at the mouthpiece end 52 of the cartridge except for a mouthpiece opening/aerosol outlet 60, from the mouthpiece 33, which is in fluid communication with the housing inner tube 62. The housing part 32 includes an opening in a sidewall which provides the air inlet 50 for the cartridge. The air inlet 50 in this example has an area of around 2 mm². The outer surface of the outer wall 64 of the housing part 32 includes the protrusions 56 discussed above which engage with corresponding detents in the interior surface of the receptacle wall 12 defining the receptacle 8 to provide a releasable mechanical engagement between the cartridge and the aerosol provision device. The inner surface of the outer wall 64 of the housing part includes further protrusions 66 which act to provide an abutment stop for locating the dividing wall element 36 along the longitudinal axis L when the cartridge is assembled. The outer wall 64 of the housing part 32 further comprises holes which provide latch recesses 68 arranged to receive corresponding latch projections 70 in the end cap to fix the end cap to be housing part when the cartridge is assembled.

The outer wall 64 of the housing part 32 includes a double-walled section 74 that defines a gap 76 in fluid communication with the air inlet 50. The gap 76 provides a portion of the air channel through the cartridge. In this example the doubled-walled section 74 of the housing part 32 is arranged so the gap defines an air channel running within the housing outer wall 64 parallel to the longitudinal axis with a cross-section in a plane perpendicular to the longitudinal axis of around 3 mm². The gap/portion of air channel 76 defined by the double-walled section of the housing part extends down to the open end of the housing part 32.

The air channel seal 34 is a silicone moulding generally in the form of a tube having a through hole 80. The outer wall of the air channel seal 34 includes circumferential ridges 84 and an upper collar 82. The inner wall of the air channel seal 34 also includes circumferential ridges, but these are not visible in FIG. 2. When the cartridge is assembled the air channel seal 34 is mounted to the housing inner tube 62 with an end of the housing inner tube 62 extending partly into the through hole 80 of the air channel seal 34. The through hole 80 in the air channel seal has a diameter of around 5.8 mm in its relaxed state whereas the end of the housing inner tube 62 has a diameter of around 6.2 mm so that a seal is formed when the air channel seal 34 is stretched to accommodate the housing inner tube 62. This seal is facilitated by the ridges on the inner surface of the air channel seal 34.

The outlet tube 38 comprises a tubular section, for instance made of ANSI 304 stainless steel or polypropylene, with an internal diameter of around 8.6 mm and a wall thickness of around 0.2 mm. The bottom end of the outlet tube 38 includes a pair of diametrically opposing slots 88 with an end of each slot having a semi-circular recess 90. When the cartridge is assembled the outlet tube 38 mounts to the outer surface of the air channel seal 34. The outer diameter of the air channel seal is around 9.0 mm in its relaxed state so that a seal is formed when the air channel seal 34 is compressed to fit inside the outlet tube 38. This seal is facilitated by the ridges 84 on the outer surface of the air channel seal 34. The collar 80 on the air channel seal 34 provides a stop for the outlet tube 38.

The aerosolisable material transport element 42 comprises a capillary wick and the vaporiser (aerosol generator) 40 comprises a resistance wire heater wound around the capillary wick. In addition to the portion of the resistance wire wound around the capillary wick, the vaporiser comprises electrical leads 41 which pass through holes in the plug 44 to contact electrodes 46 mounted to the end cap 54 to allow power to be supplied to the vaporiser via the electrical interface the established when the cartridge is connected to an aerosol provision device. The vaporiser leads 41 may comprise the same material as the resistance wire wound around the capillary wick, or may comprise a different material (e.g. lower-resistance material) connected to the resistance wire wound around the capillary wick. In this example the heater coil 40 comprises a nickel iron alloy wire and the wick 42 comprises a glass fibre bundle. The vaporiser and aerosolisable material transport element may be provided in accordance with any conventional techniques and is may comprise different forms and/or different materials. For example, in some implementations the wick may comprise fibrous or solid a ceramic material and the heater may comprise a different alloy. In other examples the heater and wick may be combined, for example in the form of a porous and a resistive material. More generally, it will be appreciated the specific nature aerosolisable material transport element and vaporiser is not of primary significance to the principles described herein.

When the cartridge is assembled, the wick 42 is received in the semi-circular recesses 90 of the outlet tube 38 so that a central portion of the wick about which the heating coil is would is inside the outlet tube while end portions of the wick are outside the outlet tube 38.

The plug 44 in this example comprises a single moulding of silicone, may be resilient. The plug comprises a base part 100 with an outer wall 102 extending upwardly therefrom (i.e. towards the mouthpiece end of the cartridge). The plug further comprises an inner wall 104 extending upwardly from the base part 100 and surrounding a through hole 106 through the base part 100.

The outer wall 102 of the plug 44 conforms to an inner surface of the housing part 32 so that when the cartridge is assembled the plug in 44 forms a seal with the housing part 32. The inner wall 104 of the plug 44 conforms to an inner surface of the outlet tube 38 so that when the cartridge is assembled the plug 44 also forms a seal with the outlet tube 38. The inner wall 104 includes a pair of diametrically opposing slots 108 with the end of each slot having a semi-circular recess 110. Extended outwardly (i.e. in a direction away from the longitudinal axis of the cartridge) from the bottom of each slot in the inner wall 104 is a cradle section 112 shaped to receive a section of the aerosolisable material transport element 42 when the cartridge is assembled. The slots 108 and semi-circular recesses 110 provided by the inner wall of the plug 44 and the slots 88 and semi-circular recesses 90 of the outlet tube 38 are aligned so that the slots 88 in the outlet tube 38 accommodate respective ones of the cradles 112 with the respective semi-circular recesses in the outlet tube and plug cooperating to define holes through which the aerosolisable material transport element passes. The size of the holes provided by the semi-circular recesses through which the aerosolisable material transport element passes correspond closely to the size and shape of the aerosolisable material transport element, but are slightly smaller so a degree of compression is provided by the resilience of the plug 44. This allows aerosolisable material to be transported along the aerosolisable material transport element by capillary action while restricting the extent to which aerosolisable material which is not transported by capillary action can pass through the openings. As noted above, the plug 44 includes further openings 114 in the base part 100 through which the contact leads 41 for the vaporiser pass when the cartridge is assembled. The bottom of the base part of the plug includes spacers 116 which maintain an offset between the remaining surface of the bottom of the base part and the end cap 48. These spacers 116 include the openings 114 through which the electrical contact leads 41 for the vaporiser pass.

The end cap 48 comprises a polypropylene moulding with a pair of gold-plated copper electrode posts 46 mounted therein.

The ends of the electrode posts 44 on the bottom side of the end cap are close to flush with the interface end 54 of the cartridge provided by the end cap 48. These are the parts of the electrodes to which correspondingly aligned sprung contacts in the aerosol provision device 4 connect when the cartridge 2 is assembled and connected to the aerosol provision device 4. The ends of the electrode posts on the inside of the cartridge extend away from the end cap 48 and into the holes 114 in the plug 44 through which the contact leads 41 pass. The electrode posts are slightly oversized relative to the holes 114 and include a chamfer at their upper ends to facilitate insertion into the holes 114 in the plug where they are maintained in pressed contact with the contact leads for the vaporiser by virtue of the plug.

The end cap has a base section 124 and an upstanding wall 120 which conforms to the inner surface of the housing part 32. The upstanding wall 120 of the end cap 48 is inserted into the housing part 32 so the latch projections 70 engage with the latch recesses 68 in the housing part 32 to snap-fit the end cap 48 to the housing part when the cartridge is assembled. The top of the upstanding wall 120 of the end cap 48 abuts a peripheral part of the plug 44 and the lower face of the spacers 116 on the plug also abut the base section 124 of the plug so that when the end cap 48 is attached to the housing part it presses against the resilient part 44 to maintain it in slight compression.

The base portion 124 of the end cap 48 includes a peripheral lip 126 beyond the base of the upstanding wall 112 with a thickness which corresponds with the thickness of the outer wall of the housing part at the interface end of the cartridge. The end cap also includes an upstanding locating pin 122 which aligns with a corresponding locating hole 128 in the plug to help establish their relative location during assembly.

The dividing wall element 36 comprises a single moulding of polypropylene and includes a dividing wall 130 and a collar 132 formed by projections from the dividing wall 130 in the direction towards the interface end of the cartridge. The dividing wall element 36 has a central opening 134 through which the outlet tube 38 passes (i.e. the dividing wall is arranged around the outlet tube 38). In some embodiments, the dividing wall element 36 may be integrally formed with the outlet tube 38. When the cartridge is assembled, the upper surface of the outer wall 102 of the plug 44 engages with the lower surface of the dividing wall 130, and the upper surface of the dividing wall 130 in turn engages with the projections 66 on the inner surface of the outer wall 64 of the housing part 32. Thus, the dividing wall 130 prevents the plug from being pushed too far into the housing part 32—i.e. the dividing wall 130 is fixedly located along the longitudinal axis of the cartridge by the protrusions 66 in the housing part and so provides the plug with a fixed surface to push against. The collar 132 formed by projections from the dividing wall includes a first pair of opposing projections/tongues 134 which engage with corresponding recesses on an inner surface of the outer wall 102 of the plug 44. The protrusions from the dividing wall 130 further provide a pair of cradle sections 136 configured to engage with corresponding ones of the cradle sections 112 in the part 44 when the cartridge is assembled to further define the opening through which the aerosolisable material transport element passes.

When the cartridge 2 is assembled an air channel extending from the air inlet 50 to the aerosol outlet 60 through the cartridge is formed. Starting from the air inlet 50 in the side wall of the housing part 32, a first section of the air channel is provided by the gap 76 formed by the double-walled section 74 in the outer wall 64 of the housing part 32 and extends from the air inlet 50 towards the interface end 54 of the cartridge and past the plug 44. A second portion of the air channel is provided by the gap between the base of the plug 44 and the end cap 48. A third portion of the air channel is provided by the hole 106 through the plug 44. A fourth portion of the air channel is provided by the region within the inner wall 104 of the plug and the outlet tube around the vaporiser 40. This fourth portion of the air channel may also be referred to as an aerosol/aerosol generation region, it being the primary region in which aerosol is generated during use. The air channel from the air inlet 50 to the aerosol generation region may be referred to as an air inlet section of the air channel. A fifth portion of the air channel is provided by the remainder of the outlet tube 38. A sixth portion of the air channel is provided by the outer housing inner tube 62 which connects the air channel to the aerosol outlet 60, which is located at an end of the mouthpiece 33. The air channel from the aerosol generation region to be the aerosol outlet may be referred to as an aerosol outlet section of the air channel.

Also, when the cartridge is assembled a reservoir 31 for aerosolisable material is formed by the space outside the air channel and inside the housing part 32. This may be filled during manufacture, for example through a filling hole which is then sealed, or by other means. The specific nature of the aerosolisable material, for example in terms of its composition, is not of primary significance to the principles described herein, and in general any conventional aerosolisable material of the type normally used in electronic cigarettes may be used. The present disclosure may refer to a liquid as the aerosolisable material, which as mentioned above may be a conventional e-liquid. However, the principles of the present disclosure apply to any aerosolisable material which has the ability to flow, and may include a liquid, a gel, or a solid, where for a solid a plurality of solid particles may be considered to have the ability to flow when considered as a bulk.

The reservoir is closed at the interface end of the cartridge by the plug 44. The reservoir includes a first region above the dividing wall 130 and a second region below the dividing wall 130 within the space formed between the air channel and the outer wall of the plug. The aerosolisable material transport element (capillary wick) 42 passes through openings in the wall of the air channel provided by the semi-circular recesses 108, 90 in the plug 44 and the outlet tube 38 and the cradle sections 112, 136 in the plug 44 and the dividing wall element 36 that engage with one another as discussed above. Thus, the ends of the aerosolisable material transport element extend into the second region of the reservoir from which they draw aerosolisable material through the openings in the air channel to the vaporiser 40 for subsequent vaporisation.

In normal use, the cartridge 2 is coupled to the aerosol provision device 4 and the aerosol provision device activated to supply power to the cartridge via the contact electrodes 46 in the end cap 48. Power then passes through the connection leads 41 to the vaporiser 40. The vaporiser is thus electrically heated and so vaporises a portion of the aerosolisable material from the aerosolisable material transport element in the vicinity of the vaporiser. This generates aerosol in the aerosol generation region of the air path. Aerosolisable material that is vaporised from the aerosolisable material transport element is replaced by more aerosolisable material drawn from the reservoir by capillary action. While the vaporiser is activated, a user inhales on the mouthpiece end 52 of the cartridge. This causes air to be drawn through whichever aerosol provision device air inlet 14 aligns with the air inlet 50 of the cartridge (which will depend on the orientation in which the cartridge was inserted into the aerosol provision device receptacle 8). Air then enters the cartridge through the air inlet 50, passes along the gap 76 in the double-walled section 74 of the housing part 32, passes between the plug 44 and the end cap 48 before entering the aerosol generation region surrounding the vaporiser 40 through the hole 106 in the base part 100 of the plug 44. The incoming air mixes with aerosol generated from the vaporiser to form a condensation aerosol, which is then drawn along the outlet tube 38 and the housing part inner 62 before exiting through the mouthpiece outlet/aerosol outlet 60 for user inhalation.

From the above FIGS. 1-6B, it can be seen a possible embodiment construction of aerosol provision system 1 which is configured for generating an aerosol, which is suitable for use in the context of the present disclosure (alongside potentially other forms of aerosol provision system).

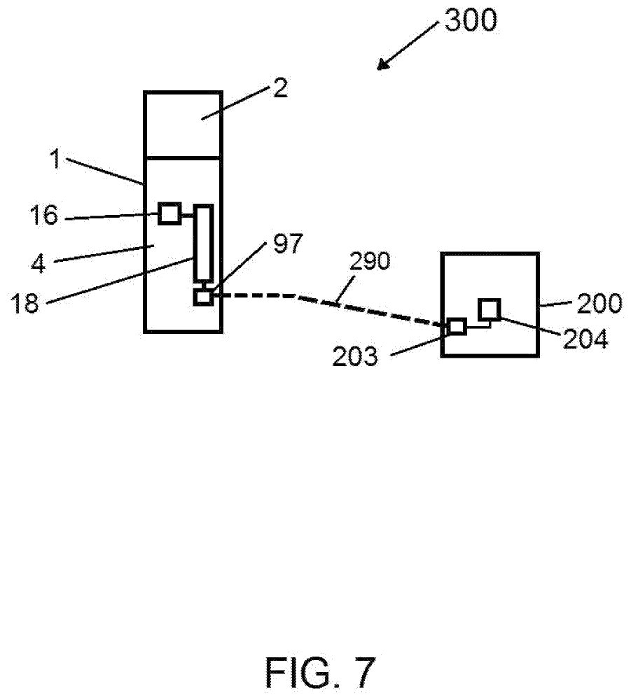

Turning now to FIGS. 7-10, in accordance with some embodiments, the present disclosure also provides methods, and corresponding systems 300, for an aerosol provision system 1 (which could be based off the aerosol provision system 1 as shown in FIGS. 1-6B, for instance—although other forms of aerosol provisions system could appreciably be also used, in so far as they are able to generate an aerosol).

At a general level, the method may comprise generating data from the aerosol provision system 1; and then transmitting, such as wirelessly transmitting in accordance with some narrower embodiments, the data to a third party location 200, where the data is then received at the third party location 200. In this way therefore, and again at a general level, the present disclosure may thus provide for mechanisms for transmitting data from the aerosol provision system 1 to such a third party location/device 200.

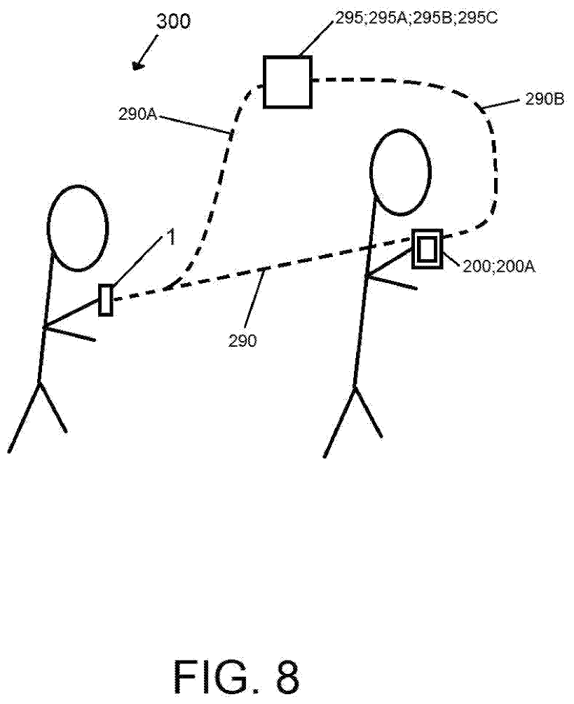

By the phrase ‘third party’ as used herein, this may be interpreted as covering locations and devices which the user/owner of the aerosol provision system is not intended to be able to routinely access. Accordingly, such a third party location may appreciably not comprise an electrical device (such as a mobile phone) which belongs to the user of the aerosol provision system 1. Thus instead, such a third party location 200 in accordance with some embodiments could comprise an electrical device 200A which does not belong to the user, such as instead an electrical, and/or portable, device belonging to a friend, or family member of the user (such as a parent, a grandparent, a spouse, or other relative, of the user of the aerosol provision system). Such embodiments are related to the embodiment of FIG. 8, as will be described. In this way, the friend or family member may be able to receive the data transmitted by the aerosol provision system, for monitoring purposes, or so they can react on the data, as will be described.

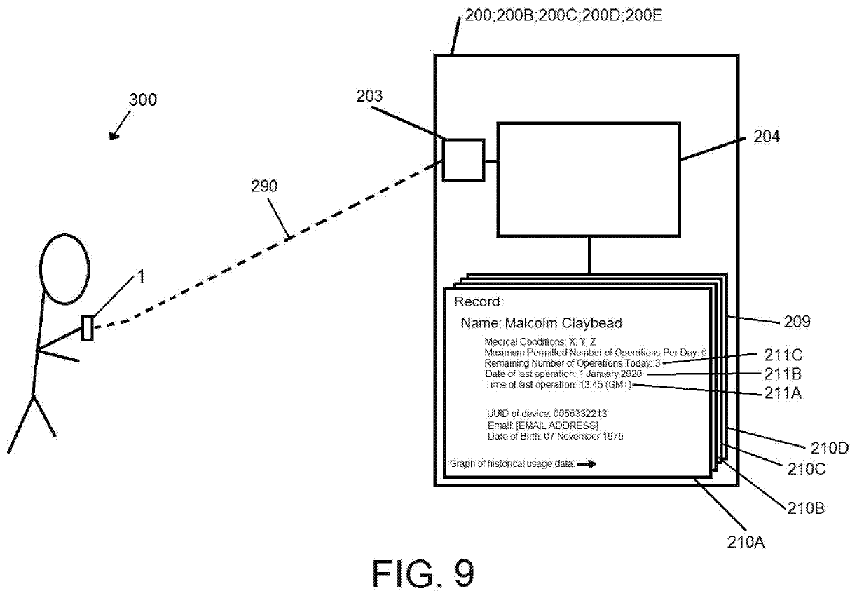

With respect to any such third party location, this may also be interpreted as covering other locations and devices which may typically have a desire to have access to the data from the aerosol provision system, and which are again locations which the user of the aerosol provision system is not intended to be able to otherwise routinely access. Such a third party location 200 may thus also comprise a health provider 200B; an insurance provider 200C; a manufacturer 200D of the aerosol provision system; and/or even a supplier 200E of the aerosol provision system 1 (as is shown in the embodiment from FIG. 9, for instance).

In this regard, it should be appreciated that the precise nature of the data that is transmitted to the third party may vary with the third party location that the data is intended to be transmitted to. For example, personal information, such as a user identifier or any information around the condition or health of the user, may only be transmitted to a health provider 200B. In these instances, such information may be transmitted only with the explicit authorisation of the user. In other instances, the data to be transmitted may be anonymised (that is to say, have a link to the specific user themselves). For example, device diagnostics or the like, may be sent anonymously, even with respect to the device itself.

Appreciating the foregoing disclosure of what encompasses a ‘third party’, to facilitate potentially real-time monitoring of the aerosol provision system, in accordance with some embodiments, the aerosol provision system 1 may be configured to generate the data, and transmit the data to the third party location 200, whilst the aerosol provision system 1 is generating an aerosol (for instance using the aerosol generator 40 described previously). In this way, by transmitting the data whilst the aerosol provision system 1 is still generating the aerosol, the data may be acted upon that much sooner, which may be preferable in accordance with some embodiments. This might be the case, for instance, in embodiments where following the data being received at the third party location 200, the third party location 200 is then configured to wirelessly transmit a response signal back to the aerosol provision system 1, which is a response signal which is then received at the aerosol provision system 1.

A potential application of this, in a very particular embodiment, could be in the embodiment of FIG. 8, where the third party location 200 comprises a portable device 200A belonging to a relative/parent of the user of the aerosol provision system 1. In this way, and in response to the user operating the aerosol provision system 1 to generate the aerosol, the data may be transmitted to the third party location 200 (the portable device 200A belonging to the relative/parent of the user), if required the relative/parent may then instruct the portable device 200A to transmit the response signal back to the aerosol provision system 1. As to what such a response signal might comprise, appreciably this could be a number of different things, depending on its required application. For instance, and in accordance with some embodiments, the response signal may comprise a command to perform a predetermined operation of the aerosol provision system. In this way, and in accordance with some embodiments thereof, in response to receiving the response signal, the aerosol provision system 1 may be configured to perform (and potentially automatically perform in some narrower embodiments) the predetermined operation of the aerosol provision system 1. It will be appreciated that in embodiments where such a predetermined operation is instructed, this may be a variety of different things, such as in accordance with some embodiments the predetermined operation comprising an operation to disable, such as temporarily disable (e.g. for a predetermined period of time, and/or until a further operation is received by the aerosol provision system to re-enable) the aerosol provision system 1 and/or the aerosol generator 40 from the aerosol provision system 1. In this way, it may be seen that by way of this operation, the third party location-whether this be a relative of the user of the aerosol provision system or some other entity, such as a health provider or manufacturer of the aerosol provision system, may thus be able to control one or more operations of the aerosol provision system 1. This might be because, in accordance with some very narrow embodiments, the parent/relative (or even a health or insurance provider) of the user of the aerosol provision system wishes to control or limit the usage of the aerosol provision system by the user who the parent/relative/provider is responsible for, or it could be because a manufacturer or supplier of the aerosol provision system may, in response to receiving the data from the aerosol provisions system, determine that the aerosol provision system 1 is faulty and/or is being used in a way it should not. Thus again, in these instances, the manufacturer or supplier of the aerosol provision system 1 may transmit the response signal back to the aerosol provision system 1, to potentially control the operation of the aerosol provision system 1.

Appreciably however, where such a predetermined operation is employed, this could equally in accordance with some embodiments comprise a notification which is configured to be output, or displayed, by the aerosol provision system 1. For instance, in accordance with some embodiments, the notification may be displayed on a display of the aerosol provision system 1, and/or could comprise a notification in a very particular embodiment which instructs the user of the aerosol provision system to stop using the aerosol provision system. Equally, in accordance with some embodiments, the notification may comprise a suggestion to operate the aerosol provision system in a predetermined way.

Noting the above therefore, it may be seen that by virtue of the aerosol provision system 1 generating the data (such as usage data), and the aerosol provision system 1 then transmitting this data to the third party location, the third party location may be able to act on this received data, e.g. to log it somewhere, or to act on the data through the sending back of a response signal to the aerosol provision system 1.

With respect to the transmission of the data from the aerosol provision system 1, it is envisaged in some embodiments that the aerosol provision system 1 may be configured to wirelessly transmit the data 290 directly to the third party location/device 200. For instance, yet although not necessarily, in a particularly convenient embodiment, the data 290 from the aerosol provision system 1 may be wirelessly transmitted by the aerosol provision system 1, as a signal, by Bluetooth®, or via a Wi-Fi connection in accordance with some embodiments.

Appreciably however, it is envisaged that the aerosol provision system 1 herein provided may, in accordance with some embodiments (such as the embodiment from FIG. 8), further comprise an intermediary device 295, or second device 295 (such as a router 295A, Wi-Fi router 295B), or potentially even a second aerosol provision system 295C, for conveying the signal 290 from the aerosol provision system 1 to the third party location/device 200. Appreciably, any such intermediary device 295 may be then configured to receive the data 290A from the aerosol provision system 1, and be configured to transmit the data (see data 290B from FIG. 8) as part of a signal to the third party location 200. This may be the case, for instance, in the particular embodiment of FIG. 8, where the data 290 may be transmitted either directly, or via a Wi-Fi router 295B, to a third party location 200 which comprises the portable device 200A of a relative of the user of the aerosol provision system 1. In this way therefore, and in accordance with some embodiments, it may be appreciated that the third party location/device 200 may be located in, or part of, a wireless network which is connected to the aerosol provision system 1 and the third party location/device 200.

With regards to the third party location 200 which is associated with the aerosol provision system 1, as noted previously this third party location 200 may appreciably be a number of different things, depending on the intended application of the system 300. This being said, and in accordance with some embodiments, prior to generating the data from the aerosol provision system 1, the aerosol provision system may be configured to determine the third party location.

Appreciably, this step of the aerosol provision system 1 determining the third party location 200 could be achieved by the aerosol provision system 1 retrieving data from a memory (e.g. as part of any employed control circuitry 18 from the aerosol provision system) of the aerosol provision system which identifies the identity of the third party location. Any such data from the memory could appreciably be pre-loaded into the aerosol provision system 1, or could be supplied as part of an initial setup process or pairing process between the aerosol provision system 1 and the third party location 200 (or some other device).

Equally however, in accordance with some embodiments, the aerosol provision system may be configured to determine the third party location in response to a selection of the third party location by the user of the aerosol provision system. In this way for instance, in so far as the third party location 200 may comprise a health provider 200B or an insurance provider 200C for instance, the aerosol provision system 1 may be configured to allow the details of the third party location 200 to be added to the aerosol provision system, such as to any memory thereof in a particular embodiment, for allowing the aerosol provision system 1 to determine the third party location as noted above.

Thus in so far as any particular third party location 200 has been associated with the aerosol provision system 1, as noted previously the third party location 200 may be configured to receive the data which is wirelessly transmitted from the aerosol provision system, when the aerosol provision system is in use.

Turning now to further potential embodiments, such as those relating to FIG. 9 for instance, in accordance with some of these embodiments, following the data being received at the third party location, the data may be processed by the third party location 200 to determine the identity of the aerosol provision system 1. This may be achieved, for instance, through there being provided identifier information in the data which is transmitted, e.g. through way of there being a product ID, or some other universally unique identifier (UUID), which is contained within the data which is transmitted as part of a signal from the aerosol provision system comprising the data.

Once the identity of the aerosol provision system 1 has been determined, the third party location may then in accordance with some embodiments be configured to determine the identity of a person which is associated with the identity of the aerosol provision system. This might particularly be the case, in accordance with some embodiments, where the third party location 200 comprises any of a health provider 200B; an insurance provider 200C; a manufacturer 200D of the aerosol provision system; and/or even a supplier 200E of the aerosol provision system 1.

This determination step may be achieved, in a very particular embodiments for instance, through an earlier pairing or association of the aerosol provision system 1 (including any product ID, or UUID) with a record 210A relating to the person, wherein the record 210A is located in a database 209 comprising a plurality of records 210A;210B;210C;210D. In this way, and once the data 290 is received at the third party location, in some variants of this particular embodiment, the third party location 200 may be configured to search the database to match the identifier information in the data 290 against a record from the database. In the particular example of FIG. 8, the data 290 may be seen as comprising a UUID for the aerosol provision system 1 of ‘0056332213’ which is determined as matching against a record 210A from the database 209 to a particular person called ‘Malcolm Claybead’.

Thus in so far as the identity of the aerosol provision system 1 has been determined, and the third party location 200 then determines the identity of a person which is associated with the identity of the aerosol provision system, in accordance with some embodiments, the third party location 200 may be configured to modify at least one record entry 211 which is associated with the person and/or the record 210A. As to what such a modification might comprise, appreciably this may depend on the content of the data 290 which is transmitted from the aerosol provision system.

For instance, and with reference to the embodiment from FIG. 9 as an example, in accordance with some embodiments, the at least one record entry 211 may comprise an entry 211A indicating the time when the aerosol provision system 1 was last used, and/or an entry 211B indicating the date when the aerosol provision system 1 was last used. The at least one record entry could equally comprise a counter or parameter 211C relating to an operation of the aerosol provision system.

With reference to the above applications therefore, it may be appreciated that in accordance with some embodiments, the data 290 which is wirelessly transmitted to the third party location by the aerosol provision system 1 may comprise usage data relating to the aerosol provision system. This usage data could comprise a number of different things, such as data indicating the time or date when the aerosol provision system was last used, and/or could comprise data indicating another parameter of operation of the aerosol provision system, such as the number of times the aerosol provision system and/or its aerosol generator 40 has been operated, and/or the number of times the aerosol provision system and/or its aerosol generator 40 has been operated in a predetermined period of time (e.g. a day, a week, a month, a year, or some other predetermined period of time).

Appreciably therefore, in so far as any such usage data may be comprised as part of the data 290, any such usage data may in accordance with some embodiments be used by the third party location 200 to modify the record entry 211. Equally, in accordance with some embodiments, modifying the record entry may comprise the third party location 200 adding at least some, or all, of this usage data to the record entry 211.

From the above, it can therefore be seen that the third party location 200 may be configured to conveniently receive the data 290 from the aerosol provision system 1, and then use this data 290 in some cases to keep a record relating to that aerosol provision system 1, or a person which is associated with the aerosol provision system 1, updated. Through such updates, it may be seen that these updates may then serve to allow the third party location 200, as required, to wirelessly transmit a response signal back to the aerosol provision system (as described previously above), such to potentially instruct the aerosol provision system to perform a predetermined operation (e.g. to turn off or disable the aerosol generator 40 of the aerosol provision system). Practical implementations for this functionality might be, for instance, in any of the following embodiments as illustrative examples:
i) where the third party location 200 comprises a health provider 200B, and where the received data comprises usage data which indicates the aerosol generator 40, or the aerosol provision system 1, as having been operated, for generating an aerosol, more than a predetermined amount in a predetermined amount of time, the health provider may instruct the aerosol provision system to perform a predetermined operation (e.g. to turn off or disable the aerosol generator 40 of the aerosol provision system) to prevent excessive usage of the aerosol provision system. This might appreciably be especially the case, where the active constituent of the aerosol comprises a cannabinoid, such that the health provider may wish to prevent over-generation of the aerosol.
ii) where the third party location 200 comprises a manufacturer 200D of the aerosol provision system, and where the received data 290 comprises data which indicates the aerosol generator 40, or the aerosol provision system 1, as having overheated or developed a fault, the manufacturer 200D may instruct the aerosol provision system to perform a predetermined operation (e.g. to turn off or disable the aerosol generator 40 of the aerosol provision system) to prevent further damage to the aerosol provision system 1. Equally/alternatively, the manufacturer 200D may instruct the aerosol provision system to perform a predetermined operation, such as in some embodiments to output a notification from the aerosol provision system. Such a notification in accordance with some particular embodiments thereof may comprise a notification which informs the user of the fault, and/or a notification which instructs the user to proceed to a predetermined location to have the aerosol provision system 1 assessed for potential repair.
iii) where the third party location 200 comprises a supplier 200E of the aerosol provision system, and where the received data 290 comprises data which indicates a remaining amount of aerosol-generating material is low or empty, the supplier 200E may instruct the aerosol provision system to perform a predetermined operation, such as in some embodiments to output a notification from the aerosol provision system, which could be a notification which includes a suggestion to operate the aerosol provision system in a predetermined way (e.g. with a new/refilled cartridge of aerosol-generating material), and/or which could be a notification which includes a request for the user to stop operating the aerosol provision system 1 (e.g. until such time as they have replaced or refilled the amount of aerosol-generating material in the aerosol provision system). In this way as well, and in so far as the third party location 200 may instruct the aerosol provision system to perform a predetermined operation, this may also help in these instances to help prevent damage to the aerosol provision system (e.g. by preventing a ‘dry-out’ situation in the aerosol provision system 1 where an insufficient amount of aerosol-generating material is remaining therein).
iv) where the third party location 200 comprises an electrical or portable device 200A which does not belong to the user of the aerosol provision system 1, such as a relative of the user of the aerosol provision system 1, the relative may be able to then track the usage of the aerosol provision system 1, such to allow the relative to react in instances where this usage is otherwise deemed adverse or inappropriate by the relative. Thus in accordance with these embodiments, the data 290 which is transmitted to the third party location 200 may help to allow better monitoring of the aerosol provision system 1, and also allow the third party location 200 to more readily oversee and prevent any potential adverse events or operations relating to the aerosol provision system 1.

Thus the herein described systems, where the data 290 can be transmitted by the aerosol provisions system to a separate device or other third party location 200 may thus consequentially provide several clear benefits over existing aerosol provisions systems where this functionality is otherwise not provided.

Appreciating the above, there may be some instances where the provision of this data by the aerosol provision system 1 to a third party location 200 may need to be temporarily, or permanently, deactivated. This might be the case, for instance, where it is determined that the battery or power consumption of the aerosol provision system 1 needs to be preserved, and/or potentially in instances where the aerosol provision system is needing to used by a different person who does not need their usage of the aerosol provision system monitored (e.g. by a different person who does not need their usage of the aerosol provision system monitored at an electrical or portable device 200A belonging to a relative). It may also be appropriate to have this data 290 transmission functionality deactivated in instances or territories where there is no apparent manufacturer 200D or supplier 200E of the aerosol provision system 1 who might otherwise be able to monitor the usage of the aerosol provision system 1.

This being the case, and in accordance with some embodiments, the aerosol provision system 1 may be configured to switch between a first mode of operation M1 in which the aerosol provision system is configured to wirelessly transmit data to a remote device or third party location 200, and a second mode of operation M2 in which the aerosol provision system 1 is configured to not wirelessly transmit the data to the device or third party location 200.

With this functionality, the aerosol provision system 1 may be configured, such as potentially prior to each operation of the aerosol provision system to generate an aerosol, to determine whether the aerosol provision system is operating in the first mode of operation or the second mode of operation, and then wirelessly transmit the data to the remote device or third party location when the aerosol provision system is operating in the first mode of operation, but not the second mode of operation.

Where such a selective system 300 is employed, where the transmitting of the data 290 can be toggled on or off, it may be appreciated that in some instances, it may be appropriate for the aerosol provision system 1 to require an authentication which authorises the aerosol provision system to switch from the first mode of operation (where the data 290 is transmitted) to the second mode of operation (where the data 290 is not transmitted). In this way, the ability to bypass the transmission of the data 290 to the remote location (e.g. by a user who is seeking to circumvent the transmission of the data 290 to this remote location) may be better obviated.

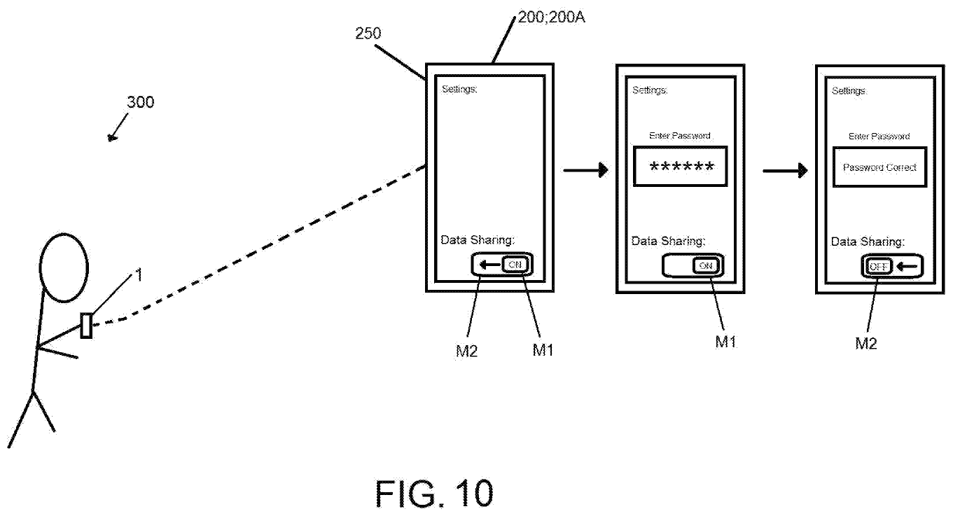

In so far as any such authentication is employed, appreciably this could comprise a number of different things, such as a password (as shown in the embodiment of FIG. 10) or a predetermined fingerprint or voice control being inputted. For inputting this authentication, this may be achieved using a device 250 which is remotely located from the aerosol provision system, such as a portable device (e.g. a mobile phone) which can be paired with the aerosol provision system 1. To be clear, any such device 250 for providing this authentication, e.g. as part of a control signal from the device 250 to the aerosol provision system 1, need not necessarily comprise the third party location/device 200 (but could be this third party location/device 200, in some embodiments), and so could instead be some other device 250 which can otherwise be paired with the aerosol provision system 1 to provide the control signal to the aerosol provision system 1. In this way, as long as the user of the aerosol provision system 1 can then provide the authentication, the aerosol provision system 1 can then use this authentication from the control signal to switch the aerosol provision system from the first mode of operation M1 to the second mode of operation M2. Otherwise, if no (or an incorrect) authentication is provided, the aerosol provision system 1 will then not switch from the first mode of operation M1 to the second mode of operation M2.

In so far as any authentication is employed, it may be appreciated in accordance with some embodiments that the authentication may only be needed to switch the aerosol provision system from the first mode of operation M1 to the second mode of operation M2, but not necessarily to switch the aerosol provision system from the second mode of operation M2 to the second mode of operation M1. However, switching the aerosol provision system 1 from the second mode of operation M2 to the first mode of operation M1 could require authentication in accordance with some embodiments, if needed.

Appreciating the foregoing therefore, there has accordingly been described a method for an aerosol provision system, wherein the method comprises:
generating data from the aerosol provision system;
wirelessly transmitting the data to a third party location; and
receiving the data at the third party location.

There has also been described a communication system comprising:
an aerosol provision system, for generating an aerosol, and configured to wirelessly transmit data in a first mode of operation; and
a device, remotely located from the aerosol provision system, wherein the device is configured to transmit a control signal to the aerosol provision system for instructing the aerosol provision system to change between:
   the first mode of operation in which the aerosol provision system is configured to wirelessly transmit data; and
   a second mode of operation in which the aerosol provision system is configured to not wirelessly transmit the data.

There has also been described a communication system comprising:
an aerosol provision system, for generating an aerosol, and configured to generate, and wireless transmit, data; and
a third party location, wherein the third party location is configured to receive the data from the aerosol provision system.

There has also been described an aerosol provision system for generating an aerosol, wherein the aerosol provision system is configured to:
operate in a first mode of operation in which the aerosol provision system is configured to wirelessly transmit data to a predetermined third party location; and
operate in a second mode of operation in which the aerosol provision system is configured to not wirelessly transmit data to the predetermined third party location.

There has also been described a method for an aerosol provision system 1, wherein the method comprises: generating data, such as usage data, from the aerosol provision system 1. The data 290 is then wirelessly transmitted by the aerosol provision system 1, as part of a wireless signal, and is received at a third party location 200. Such a third party location could be an electrical or portable device 200A belonging to a relative of the user of the aerosol provision system 1. In this way, the relative, or other third party, can potentially send back a response signal to the aerosol provision system 1 for controlling an operation of the aerosol provision system 1.

In order to address various issues and advance the art, this disclosure shows by way of illustration various embodiments in which the claimed invention(s) may be practiced. The advantages and features of the disclosure are of a representative sample of embodiments only, and are not exhaustive and/or exclusive. They are presented only to assist in understanding and to teach the claimed invention(s). It is to be understood that advantages, embodiments, examples, functions, features, structures, and/or other aspects of the disclosure are not to be considered limitations on the disclosure as defined by the claims or limitations on equivalents to the claims, and that other embodiments may be utilised and modifications may be made without departing from the scope of the claims. Various embodiments may suitably comprise, consist of, or consist essentially of, various combinations of the disclosed elements, components, features, parts, steps, means, etc. other than those specifically described herein, and it will thus be appreciated that features of the dependent claims may be combined with features of the independent claims in combinations other than those explicitly set out in the claims. The disclosure may include other inventions not presently claimed, but which may be claimed in future.

Purely for completeness, with respect to any employed embodiments where the aerosol provision system 1 may be configured to wirelessly transmit the data 290, e.g. as part of a signal, to the third party location 200, it may be appreciated that in accordance with such embodiments, the aerosol provision system 1 and the corresponding third party location 200 may be configured appropriately to effect this data transfer.

For instance, in accordance with some of these embodiments, the aerosol provision system 1 may comprise a transmitter 96, connected to the control circuitry 18 of the aerosol provision system 1, for transmitting the data 290. In this way, and as required, the third party location 200 may then potentially also comprise a receiver 203 for receiving the data 290. Where appropriate as well, the third party location 200 in accordance with some embodiments may be also provided with a controller or control circuitry 204, for receiving the data 290 from the receiver 203, for performing any of the operations as herein described, such as for controlling the sending back of the response signal to the aerosol provision system 1, and/or for the controlling of any interactions with, or relating to, the database 209.

Equally, and for completeness as well, in so far as any other forms of wireless communication are employed, such as Bluetooth®, a corresponding Bluetooth pairing arrangement may appreciably be provided between the aerosol provision system 1 and the third party location 200 (and potentially also between any intermediary device 295 in the case of embodiments along the lines of the embodiment shown in FIG. 8).

1. A method for an aerosol provision system, wherein the method comprises:
generating data from the aerosol provision system;
wirelessly transmitting the data to a third party location; and
receiving the data at the third party location.

2. A method according to claim 1, wherein generating data from the aerosol provision system, and transmitting the data to the third party location, are performed whilst the aerosol provision system is generating an aerosol.

3. A method according to claim 1, wherein the method further comprises:
following the data being received at the third party location, wirelessly transmitting a response signal back to the aerosol provision system; and
receiving the response signal at the aerosol provision system.

4. A method according to claim 3, wherein the response signal comprises a command to perform a predetermined operation of the aerosol provision system, and the method further comprises:
in response to receiving the response signal, performing the predetermined operation of the aerosol provision system.

5. A method according to claim 4, wherein the predetermined operation comprises disabling the aerosol provision system.

6. A method according to claim 4, wherein the predetermined operation comprises disabling an aerosol generator from the aerosol provision system.

7. A method according to claim 4, wherein the predetermined operation comprises outputting a notification from the aerosol provision system.

8. A method according to claim 4, wherein the predetermined operation comprises displaying a notification from the aerosol provision system.

9. (canceled)

10. (canceled)

11. A method according to claim 1, wherein the method further comprises:
prior to generating the data from the aerosol provision system, configuring the aerosol provision system to determine the third party location.

12. A method according to claim 1, wherein the third party location comprises a portable device.

13. (canceled)

14. A method according to claim 12, wherein the portable device is part of a wireless network which is connected to the aerosol provision system and the portable device.

15. A method according to claim 1, wherein the third party location comprises a second aerosol provision system.

16-19. (canceled)

20. A method according to claim 1, wherein following the data being received at the third party location, the method further comprises:
processing the data to determine an identity of the aerosol provision system;
using the identity of the aerosol provision system to determine the identity of a person which is associated with the identity of the aerosol provision system; and
modifying a record entry which is associated with the person.

21. A method according to claim 1, wherein the data which is wirelessly transmitted to the third party location comprises usage data relating to the aerosol provision system.

22. A method according to claim 21, when further dependent on claim 20, wherein modifying the record entry comprises using the usage data to modify the record entry.

23. A method according to claim 21, when further dependent on claim 20, wherein modifying the record entry comprises adding the usage data to the record entry.

24. A method according to claim 1, wherein the method comprises:
determining whether the aerosol provision system is operating in a first mode of operation or a second mode of operation; and
wirelessly transmitting the data to the third party location when the aerosol provision system is operating in the first mode of operation, but not the second mode of operation.

25. A method according to claim 24, wherein the method further comprises:
requiring an authentication to switch the aerosol provision system from the first mode of operation to the second mode of operation.

26. (canceled)

27. A communication system comprising:
an aerosol provision system, for generating an aerosol, and configured to wirelessly transmit data in a first mode of operation; and
a device, remotely located from the aerosol provision system, wherein the device is configured to transmit a control signal to the aerosol provision system for instructing the aerosol provision system to change between:
the first mode of operation in which the aerosol provision system is configured to wirelessly transmit data; and
a second mode of operation in which the aerosol provision system is configured to not wirelessly transmit the data.

28-32. (canceled)

33. A communication system comprising:
an aerosol provision system, for generating an aerosol, and configured to generate, and wireless transmit, data; and
a third party location, wherein the third party location is configured to receive the data from the aerosol provision system.

34-39. (canceled)
